(19) United States
(12) Patent Application Publication
(10) Pub. No.: US 2025/0025482 A1
(43) Pub. Date: Jan. 23, 2025

(54) USE OF CANNABIDIOL IN THE TREATMENT OF EPILEPSY

(71) Applicant: GW Research Limited, Histon Cambridge (GB)

(72) Inventor: Volker KNAPPERTZ, Cambridge (GB)

(21) Appl. No.: 18/292,844

(22) PCT Filed: Jul. 27, 2022
(86) PCT No.: PCT/GB2022/051961

§ 371 Date: Jan. 26, 2024

Related U.S. Application Data
(60) Provisional application No. 63/226,466, filed on Jul. 28, 2021.

(51) Int. Cl. A61K 31/00; A61P 25/12

(52) U.S. Cl. CPC A61K 31/658; A61P 25/12

(57) ABSTRACT

The present disclosure relates to the use of cannabidiol (CBD) for the treatment of seizures in patients aged about 1-2 years old. In particular the CBD appears particularly effective in reducing seizures in patients suffering with etiologies that include: Lennox-Gastaut Syndrome; Tuberous Sclerosis Complex; Dravet Syndrome; Doose Syndrome; Aicardi syndrome; CDKL5 and Dun15q.

The present application is related to, and claims the benefit of, U.S. 63/226,466 filed on 28 Jul. 2021 (Jul. 28, 2021), the contents of which are hereby incorporated by reference in their entirety.

### BACKGROUND TO THE INVENTION

Epilepsy occurs in approximately 1% of the population worldwide, (Thurman et al., 2011) of which 70% are able to adequately control their symptoms with the available existing anti-epileptic drugs (AED). However, 30% of this patient group, (Eadie et al., 2012), are unable to obtain seizure freedom from the AED that are available and as such are termed as suffering from intractable or “treatment-resistant epilepsy” (TRE). Oftentimes, TRE arises in children during the first few years of life. The frequent, uncontrolled seizures caused by TRE lead to neurological damage which causes cognitive, behavioral, and motor delays.

Treatment of patients who develop TRE within their first few years of life (e.g, patients between 1 and 2 years of age) is challenging, because these patients exhibit physiological differences as compared to older children and adults. (Fabiano et al. (2012). Expert Opinion on Drug Safety, 11:1, 95-105.) For example, channels, transporters, and enzymes (e.g., the CYP enzymes) are subjected to developmental maturation, renal function is immature, gastric intraluminal pH is higher, gastric emptying is delayed, and protein-binding capacity is not as efficient as in adults. These physiological differences lead to differences in the bioavailability and absorption of drugs as compared to older children and adults. For example, while benzodiazepines are an effective antiseizure drug for older patients, they cause paradoxical seizures in children s 2 years of age (Ben-Ari Y. Crit Rev Neurobiol 2006; 18:135-144).

Adverse drug reactions (ADR) are frequently reported in children <2 years old. A 10-year surveillance study considering ADRs in children and adolescents s 18 years or age found that 52% of the reports were observed in children s 2 years of age (Aagard et al. Drug Saf 2010; 33:327-39).

There exists a need in the art for improved compositions and methods for treating TRE in patients between 1-2 years of age.

### BRIEF SUMMARY OF THE DISCLOSURE

Provided herein is a method of treating seizures using cannabidiol (CBD), which significantly shortens the dose titration period. This results in patients receiving maintenance doses sooner after initiating treatment, which reduces the occurrence of breakthrough seizures and improves adherence to treatment.

In some embodiments, the method of the disclosure, comprises administering to the patient cannabidiol (CBD) having a purity of at least 95% (w/w), wherein the patient is administered a starting dose of CBD of 5 mg/kg/day, and the starting dose is gradually increased to a dose is increased by increments of about 1-5 mg/kg. In some embodiments, the dose is increased by an amount ranging from 10 mg/kg/day to 50 mg/kg/day. In some embodiments, the dose increase may occur after about 1 week (7 days from the first administration of the starting dose). In some embodiments, the dose increase may occur within one week of administering the starting dose the dose is increased by increments of about 1-5 mg/kg. In some embodiments, the starting dose is increased about 4 to 6 days after administering the starting dose. In some embodiments, the starting dose is increased 6 days after administering the starting dose. In some embodiments, the starting dose is increased 5 days after administering the starting dose. In some embodiments, the starting dose is increased 4 days after administering the starting dose.

In some embodiments, the starting dose is increased by increments of about 5 mg/kg. In some embodiments, the starting dose is increased by increments of no more than 5 mg/kg every other day. In some embodiments, the starting dose is increased by about 5 mg/kg within about 4 to about 6 days after administering the starting dose.

In some embodiments, the dose of the CBD is increased to about 10 mg/kg/day. In some embodiments, the dose of the CBD is increased to about 12 mg/kg/day. In some embodiments, the dose of the CBD is increased to about 14 mg/kg/day. In some embodiments, the dose of CBD is increased to about 15 mg/kg/day. In some embodiments, the dose of the CBD is increased to about 16 mg/kg/day. In some embodiments, the dose of the CBD is increased to about 18 mg/kg/day. In some embodiments, the dose of the CBD is increased to about 20 mg/kg/day. In some embodiments, the dose of CBD is increased to about 25 mg/kg/day.

In some embodiments, the patient has a treatment resistant form of epilepsy. In some embodiments, the patient has Lennox-Gastaut Syndrome, Dravet Syndrome, tuberous sclerosis complex (TSC), Doose Syndrome, Aicardi syndrome, Myoclonic absence epilepsy, febrile infection related epilepsy syndrome (FIRES), Sturge Weber, CDKL5 or Dup15. In some embodiments, the patient has Lennox-Gastaut Syndrome. In some embodiments, the patient has Dravet Syndrome. In some embodiments, the patient has TSC.

In some embodiments, the seizures are convulsive seizures. In some embodiments, the seizures are atonic, tonic, tonic-clonic, myoclonic, or absence seizures. In some embodiments, the seizures are focal seizures. In some embodiments, the seizures are absence seizures. In some embodiments, the seizures are treatment resistant.

In some embodiments, the starting dose is increased to about 10 mg/kg/day, and the 10 mg/kg/day dose is further increased in weekly increments of about 5 mg/kg. In some embodiments, the dose is increased to a maximum of about 20 or about 25 mg/kg/day. In some embodiments, within one week of administering about 10 mg/kg/day, the dose is increased to about 20 mg/kg/day. In some embodiments, the dose of about 10 mg/kg/day is increased by about 5 mg/kg at a time point of 2 to 6 days after the first administration of 10 mg/kg/day. In some embodiments, the dose of about 15 mg/kg/day is increased by about 5 mg/kg at a time point of 2 to 6 days after the first administration of about 15 mg/kg/day. In some embodiments, the dose of about 20 mg/kg/day is increased by about 5 mg/kg at a time point of 2 to 6 days after the first administration of about 20 mg/kg/day. In some embodiments, the dose of about 10 mg/kg/day is increased every other day, up to a maximum of 20 or 25 mg/kg/day.

Provided herein is a method of treating seizures in a patient in need thereof, comprising administering to the patient a cannabidiol (CBD) drug substance, wherein the CBD drug substance has a purity of at least 95% (w/w) CBD, wherein the patient is about 1-2 years of age, and wherein the CBD is administered at a dose ranging from about 5 mg/kg/day to about 50 mg/kg/day.

Also provided herein is a cannabidiol (CBD) drug substance for use in treating seizures in a patient in need thereof, wherein the CBD drug substance has a purity of at least 95% (w/w) CBD, wherein the patient is about 1-2 years of age, and wherein the CBD is administered at a dose ranging from about 5 mg/kg/day to about 50 mg/kg/day.

In some embodiments, the CBD is administered at a dose of 25 mg/kg/day. In some embodiments, the CBD is administered at a dose of 20 mg/kg/day. In some embodiments, the CBD is administered at a dose of 15 mg/kg/day. In some embodiments, the CBD is administered at a dose of 10 mg/kg/day. In some embodiments, administering treats generalized seizures or focal seizures. In some embodiments, the generalized seizures comprise tonic, atonic, tonic-clonic, or absence seizures. In some embodiments, the focal seizures are focal motor seizures without impairment of consciousness or awareness, focal seizures with impairment of consciousness or awareness, or focal seizures which evolve to secondary generalized seizures. In some embodiments, administering reduces the total number of seizures. In some embodiments, the number of seizures is reduced by at least 50% compared to the number of seizures experienced during a baseline period before CBD was administered. In some embodiments, the number of generalized seizures or focal seizures is reduced by at least 50% compared to the number of seizures experienced during a baseline period before CBD was administered. In some embodiments, the number of generalized seizures is reduced by at least 50% compared to the number of seizures experienced during a baseline period before CBD was administered, wherein the generalized seizures comprise tonic, atonic, tonic-clonic, or absence seizures. In some embodiments, the number of focal seizures is reduced by at least 50% compared to the number of seizures experienced during a baseline period before CBD was administered, wherein the focal seizures are focal motor seizures without impairment of consciousness or awareness, focal seizures with impairment of consciousness or awareness, or focal seizures which evolve to secondary generalized seizures. In some embodiments, the number of seizures is reduced by at least 50% compared to the number of seizures experienced during a baseline period before CBD was administered at a dose of about 20 mg/kg/day. In some embodiments, the number of seizures is reduced by at least 50% compared to the number of seizures experienced during a baseline period before CBD was administered at a dose of about 25 mg/kg/day. In some embodiments, the CBD is present as a synthetic compound. In some embodiments, the CBD is present in a highly purified CBD extract. In some embodiments, the CBD drug substance has a purity of at least 98% (w/w) CBD. In some embodiments, the patient in need thereof is suffering from Lennox-Gastaut Syndrome (LGS); Tuberous Sclerosis Complex (TSC); Dravet Syndrome (DS); Doose Syndrome; Aicardi syndrome; CDKL5 or Dup15q. In some embodiments, the patient in need thereof is suffering from Lennox-Gastaut Syndrome (LGS). In some embodiments, the patient in need thereof is suffering from Dravet Syndrome (DS). In some embodiments, the patient in need thereof is suffering from Tuberous Sclerosis Complex (TSC).

### BRIEF DESCRIPTION OF THE DRAWINGS

FIG. 1 shows the ILAE 2010 proposal for revised terminology for organization of seizures and epilepsies.

### DEFINITIONS

Definitions of some of the terms used to describe the invention are detailed below:

The cannabinoids described in the present application are listed below along with their standard abbreviations.

TABLE 1
Cannabinoids and their abbreviations

| | | |
|---|---|---|
| CBD | Cannabidiol | 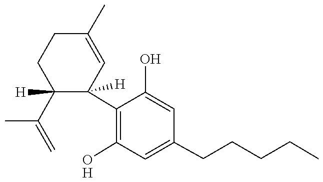 |
| CBDA | Cannabidiolic acid | 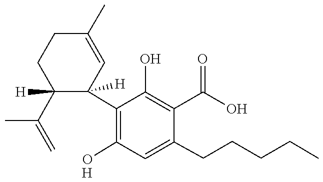 |
| CBDV | Cannabidivarin | 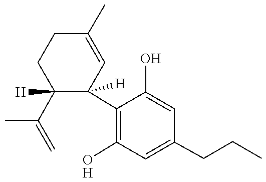 |
| CBDVA | Cannabidivarinic acid | 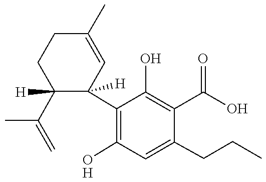 |
| THC | Tetrahydrocannabinol | 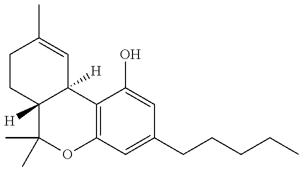 [SMILES: CCCCCc1cc(O)c2c(c1)OC(C)(C)[C@@H]1CCC(C)=C[C@@H]21] |

The table above is not exhaustive and merely details the cannabinoids which are identified in the present application for reference. So far over 60 different cannabinoids have been identified and these cannabinoids can be split into different groups as follows: Phytocannabinoids; Endocannabinoids and Synthetic cannabinoids (which may be novel cannabinoids or synthetically produced phytocannabinoids or endocannabinoids).

“Phytocannabinoids” are cannabinoids that originate from nature and can be found in the cannabis plant. The phytocannabinoids can be isolated from plants to produce a highly purified extract or can be reproduced synthetically.

“Highly purified cannabinoids” are defined as cannabinoids that have been extracted from the cannabis plant and purified to the extent that other cannabinoids and non-cannabinoid components that are co-extracted with the cannabinoids have been removed, such that the highly purified cannabinoid is greater than or equal to 95% (w/w) pure.

References to “cannabidiol” or “CBD” herein, refer to CBD that has a purity of at least 95% (w/w), unless the context clearly indicates otherwise.

“Synthetic cannabinoids” are compounds that have a cannabinoid or cannabinoid-like structure and are manufactured using chemical means rather than by the plant.

Phytocannabinoids can be obtained as either the neutral (decarboxylated form) or the carboxylic acid form depending on the method used to extract the cannabinoids. For example, it is known that heating the carboxylic acid form will cause most of the carboxylic acid form to decarboxylate into the neutral form.

“Treatment-resistant epilepsy” (TRE) or “intractable epilepsy” is defined as per the ILAE guidance of 2009 as epilepsy that is not adequately controlled by trials of one or more AED.

“Childhood epilepsy” refers to the many different syndromes and genetic mutations that can occur to cause epilepsy in childhood. Examples of some of these are as follows: Dravet Syndrome; Myoclonic-Absence Epilepsy; Lennox-Gastaut syndrome; Generalized Epilepsy of unknown origin; CDKL5 mutation; Aicardi syndrome; bilateral polymicrogyria; Dup15q; SNAP25; and febrile infection related epilepsy syndrome (FIRES); benign rolandic epilepsy; juvenile myoclonic epilepsy; infantile spasm (West syndrome); and Landau-Kleffner syndrome. The list above is non-exhaustive as many different childhood epilepsies exist.

“Atonic Seizures” are defined as a convulsive type of epileptic seizure, which causes the muscles to relax, and the patient to flop or fall.

“Mixed seizures” are defined as the existence of both generalised and focal seizures in the same patient.

The terms “50% responder” and “50% reduction in seizure” are both terms used in clinical studies. In the present application the terms define the percentage of subjects that experienced a greater than or equal to 50% reduction in the number of seizures during treatment with CBD in comparison to the number experienced during the baseline period before the CBD was administered.

The term “titration period” refers to the length of time between the starting dose of an AED and the maintenance dose.

The term “maintenance dose” refers to a dose that a patient is administered on a continuous basis (e.g., 2 weeks or more).

As used herein, “hepatic impairment” means a reduction in normal liver executory and metabolic function compared to an otherwise healthy liver. The liver is involved in the clearance of many drugs through a variety of oxidative and conjugative metabolic pathways and/or through biliary excretion of unchanged drug or metabolites. Alterations of these excretory and metabolic activities by hepatic impairment can lead to drug accumulation or, less often, failure to form an active metabolite. In some embodiments, hepatic impairment can be determined using the Child Pugh score. The Child Pugh score is described in Cholongitas, et al. “Systematic review: The model for end-stage liver disease—should it replace Child-Pugh's classification for assessing prognosis in cirrhosis?”. Alimentary Pharmacology & Therapeutics. 22 (11-22): 1079-89, which is herein incorporated by reference in its entirety.

The Child Pugh score employs five clinical measures of liver disease. Each measure is scored 1-3, with 3 indicating most severe derangement. Either the prothrombin time or INR should be used to calculate the Child-Pugh score, not both.

| Measure | 1 point | 2 points | 3 points |
|---|---|---|---|
| Total bilirubin, | (<2) | (2-3) | (>3) |
| (mg/dL) | | | |
| Serum albumin, g/dL | >3.5 | 2.8-3.5 | <2.8 |
| Prothrombin time, | <4.0 | 4.0-6.0 | >6.0 |
| prolongation (s) | | | |
| INR | <1.7 | 1.7-2.3 | >2.3 |
| Ascites | None | Mild (or suppressed | Moderate to severe |
| | | with medication) | (or refractory) |
| Hepatic | None | Grade I-II | Grade III-IV |
| encephalopathy | | | |

Chronic liver disease is classified into Child-Pugh class A to C, employing the added score from above.

| Points | Class |
|---|---|
| 5-6 | A |
| 7-9 | B |
| 10-15 | C |

In some embodiments, a patient with “mild hepatic impairment” has a Child Pugh score of A. In some embodiments, a patient with “mild hepatic impairment” has a Child Pugh score of B. In some embodiments, a patient with “mild hepatic impairment” has a Child Pugh score of C.

In some embodiments, “mild hepatic impairment” is bilirubin ≤51×the upper limit of the normal range (“ULN”) and aspartate aminotransferase (“AST”) >1×ULN, or bilirubin >1.0-1.5×ULN and any amount of AST above ULN is present. In some embodiments, “moderate hepatic impairment” is bilirubin >1.5-3.033×ULN and any amount of AST above ULN is present. In some embodiments, “severe hepatic impairment” is bilirubin ≥3.0×ULN and any amount of AST above ULN is present.

### DETAILED DESCRIPTION

### Treatment Resistant Epilepsies

In some embodiments, the compositions and methods of the disclosure are utilized to treat Intractable or treatment-resistant epilepsy (TRE). TRE as defined by the International League Against Epilepsy (ILAE) is “failure of adequate trials of two tolerated and appropriately chosen and used AED schedules (whether as monotherapies or in combination) to achieve sustained seizure freedom” (Kwan et al., 2009).

Individuals who develop epilepsy during the first few years of life are often difficult to treat and as such are often termed treatment-resistant. Children who undergo frequent seizures in childhood are often left with neurological damage, which can cause cognitive, behavioral and motor delays.

In some embodiments, the compositions and methods of the disclosure are utilized to treat childhood epilepsy. Childhood epilepsy is a relatively common neurological disorder in children and young adults with a prevalence of approximately 700 per 100,000. This is twice the number of epileptic adults per population. When a child or young adult presents with a seizure, investigations are normally undertaken in order to investigate the cause. Many different syndromes and genetic mutations can cause childhood epilepsy and as such diagnosis for these children may take some time. In some embodiments, the compositions and methods of the disclosure are utilized to treat patients 1 year of age or older. In some embodiments, the compositions and methods of the disclosure are utilized to treat patients from about 1 year of age to about 2 years of age, for example, patients that are about 12 months old, about 13 months old, about 14 months old, about 15 months old, about 16 months old, about 17 months old, about 18 months old, about 19 months old, about 20 months old, about 21 months old, about 22 months old, about 23 months old, or about 24 months old.

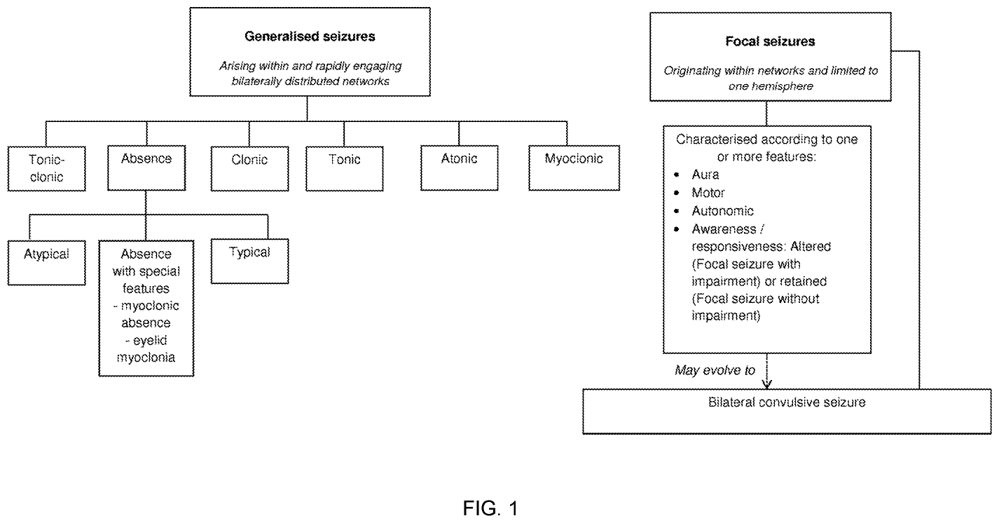

In some embodiments, the compositions and methods of the disclosure are utilized to treat repeated seizures. Repeated seizures are the main symptom of epilepsy. In some embodiments, the types of seizures a patient is experiencing are utilized to determine the type of epilepsy or the epileptic syndrome a patient is suffering from. Clinical observations and electroencephalography (EEG) tests are conducted and the type(s) of seizures are classified according to the ILAE classification described below and in FIG. 1.

The International classification of seizure types proposed by the ILAE was adopted in 1981 and a revised proposal was published by the ILAE in 2010 and has not yet superseded the 1981 classification. FIG. 1 is adapted from the 2010 proposal for revised terminology and includes the proposed changes to replace the terminology of partial with focal. In addition the term “simple partial seizure” has been replaced by the term “focal seizure where awareness/responsiveness is not impaired” and the term “complex partial seizure” has been replaced by the term “focal seizure where awareness/consciousness is impaired”.

From FIG. 1 it can be seen that Generalized seizures, where the seizure arises within and rapidly engages bilaterally distributed networks, can be split into six subtypes: Tonic-Clonic (grand mal) seizures; Absence (petit mal) Seizures; Clonic Seizures; Tonic Seizures; Atonic Seizures and Myoclonic Seizures. In some embodiments, the compositions and methods of the disclosure are used to treat generalized seizures. In some embodiments, the compositions and methods of the disclosure are used to treat tonic-clonic seizures, absence seizures, clonic seizures, tonic seizures, atonic seizures, myoclonic seizures, or combinations thereof.

Focal (partial) seizures are seizures that originate within networks limited to only one hemisphere. Focal seizures are characterized according to one or more features of the seizure, including aura, motor, autonomic and awareness/responsiveness. One type of focal seizure is a bilateral convulsive seizure. A bilateral convulsive seizure begins as a localized seizure and rapidly evolves to be distributed within bilateral networks this seizure is known as a bilateral convulsive seizure. In some embodiments, the compositions and methods of the disclosure are utilized to treat focal seizures. In some embodiments, the compositions and methods of the disclosure are utilized to treat bilateral convulsive seizures.

Focal seizures where the subject's awareness/responsiveness is altered are referred to as focal seizures with impairment and focal seizures where the awareness or responsiveness of the subject is not impaired are referred to as focal seizures without impairment. In some embodiments, the compositions and methods of the disclosure are utilized to treat focal seizures with impairment. In some embodiments, the compositions and methods of the disclosure are utilized to treat focal seizures without impairment.

Atonic seizures involve the loss of muscle tone, causing the person to fall to the ground. These are sometimes called ‘drop attacks’ and are usually brief (less than 15 seconds). Atonic seizures can occur without warning while standing, sitting and walking and the patient often suffers from trauma due to falling. In some embodiments, the compositions and methods of the disclosure are utilized to treat atonic seizures.

Atonic seizures are often associated with Lennox-Gastaut Syndrome but also occur, and may be symptomatic of other types of epileptic syndromes including: Tuberous Sclerosis Complex; Dravet Syndrome; Doose Syndrome; Aicardi syndrome; CDKL5 and Dup15q. In some embodiments, the compositions and methods of the disclosure are utilized to treat Lennox-Gastaut Syndrome, Tuberous Sclerosis Complex; Dravet Syndrome; Doose Syndrome; Aicardi syndrome; CDKL5 or Dup15q.

In some embodiments, the compositions and methods of the disclosure are utilized to treat infantile spasms.

Epileptic syndromes often present with many different types of seizure and identifying the types of seizure that a patient is suffering from is important as many of the standard AED's are targeted to treat or are only effective against a given seizure type/sub-type.

One such childhood epilepsy syndrome is Lennox-Gastaut syndrome. Lennox-Gastaut syndrome is a severe form of epilepsy. Seizures usually begin before the age of 4. Seizure types, which vary among patients, include tonic (stiffening of the body, upward deviation of the eyes, dilation of the pupils, and altered respiratory patterns), atonic (brief loss of muscle tone and consciousness, causing abrupt falls), atypical absence (staring spells), and myoclonic (sudden muscle jerks). There may be periods of frequent seizures mixed with brief, relatively seizure-free periods.

Most children with Lennox-Gastaut syndrome experience some degree of impaired intellectual functioning or information processing, along with developmental delays, and behavioural disturbances.

Lennox-Gastaut syndrome can be caused by brain malformations, perinatal asphyxia, severe head injury, central nervous system infection and inherited degenerative or metabolic conditions. In 30-35 percent of cases, no cause can be found.

The first line treatment for atonic seizures, including the treatment of atonic seizures in patients with Lennox-Gastaut syndrome usually comprises a broad spectrum AED, such as sodium valproate often in combination with lamotrigine. Other AED that may be considered include rufinamide, felbamate, clobazam and topiramate.

AED such as carbamezapine, gabapentin, oxcarbazepine, pregabalin, tiagabineor and vigabatrin are contra-indicated in atonic seizures.

Common AED defined by their mechanisms of action and titration periods are described in the following tables:

TABLE 2
Examples of narrow spectrum AED

| Narrow-spectrum | | |
|---|---|---|
| AED | Mechanism | Indication |
| Phenytoin | Sodium channel | Complex partial |
| | | Tonic-clonic |
| Phenobarbital | GABA/Calcium channel | Partial seizures |
| | | Tonic-clonic |
| Carbamazepine | Sodium channel | Partial seizures |
| | | Tonic-clonic |
| | | Mixed seizures |
| Oxcarbazepine | Sodium channel | Partial seizures |
| | | Tonic-clonic |
| | | Mixed seizures |
| Gabapentin | Calcium channel | Partial seizures |
| | | Mixed seizures |
| Pregabalin | Calcium channel | Adjunct therapy for partial seizures with or |
| | | without secondary generalisation |
| Lacosamide | Sodium channel | Adjunct therapy for partial seizures |
| Vigabatrin | GABA | Secondarily generalized tonic-clonic |
| | | seizures |
| | | Partial seizures |
| | | Infantile spasms due to West syndrome |

TABLE 3
Examples of broad spectrum AED

| Broad- | | |
|---|---|---|
| spectrum | Mech- | |
| AED | anism | Indication |
| Valproic acid | GABA/ | First-line treatment for tonic-clonic |
| | Sodium | seizures, absence seizures and |
| | channel | myoclonic seizures |
| | | Second-line treatment for partial |
| | | seizures and infantile spasms. |
| | | Intravenous use in status epilepticus |
| Lamotrigine | Sodium | Partial seizures |
| | channel | Tonic-clonic |
| | | Seizures associated with Lennox- |
| | | Gastaut syndrome |
| Topiramate | GABA/ | Seizures associated with Lennox- |
| | Sodium | Gastaut syndrome |
| | channel | |
| Zonisamide | GABA/ | Adjunctive therapy in adults with |
| | Calcium/ | partial-onset seizures |
| | Sodium | Infantile spasm |
| | channel | Mixed seizure |
| | | Lennox-Gastaut syndrome Myoclonic |
| | | Generalised tonic-clonic seizure |
| Levetiracetam | Calcium | Partial seizures |
| | channel | Adjunctive therapy for partial, |
| | | myoclonic and tonic-clonic seizures |
| Clonazepam | GABA | Typical and atypical absences |
| | | Infantile myoclonic |
| | | Myoclonic seizures |
| | | Akinetic seizures |
| | | Atonic seizures |
| Rufinamide | Sodium | Adjunctive treatment of partial |
| | channel | seizures associated with Lennox- |
| | | Gastaut syndrome |

TABLE 4
Examples of AED used specifically in childhood epilepsy

| AED | Mechanism | Indication |
|---|---|---|
| Clobazam | GABA | Adjunctive therapy in complex partial |
| | | seizures |
| | | Status epilepticus |
| | | Myoclonic |
| | | Myoclonic-absent |
| | | Simple partial |
| | | Complex partial |
| | | Absence seizures |
| | | Lennox-Gastaut syndrome |
| Stiripentol | GABA | Severe myoclonic epilepsy in infancy |
| | | (Dravet syndrome) |

TABLE 5
Titration Periods of AED

| AED | Titration Period (weeks) |
|---|---|
| Phenytoin | 3.3 |
| Levetiracetam | 4.7 |
| Valproate | 5.1 |
| Lacosamide | 5.1 |
| Carbamazepine | 5.4 |
| Topiramate | 6.1 |
| Lamotrogine | 8.1 |

In some embodiments, CBD is administered to a patient at a dose from about 1 mg/kg/day to about 25 mg/kg/day, In some embodiments, CBD is administered to a patient at a dose from about 25 mg/kg/day to about 50 mg/kg/day. For example, CBD may be administered to a patient at a dose of about 1 mg/kg/day, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 11 mg/kg/day, about 12 mg/kg/day, about 13 mg/kg/day, about 14 mg/kg/day, about 15 mg/kg/day, about 16 mg/kg/day, about 17 mg/kg/day, about 18 mg/kg/day, about 19 mg/kg/day, about 20 mg/kg/day, about 21 mg/kg/day, about 22 mg/kg/day, about 23 mg/kg/day, about 24 mg/kg/day, about 25 mg/kg/day, about 26 mg/kg/day, about 27 mg/kg/day, about 28 mg/kg/day, about 29 mg/kg/day, about 30 mg/kg/day, about 31 mg/kg/day, about 32 mg/kg/day, about 33 mg/kg/day, about 34 mg/kg/day, about 35 mg/kg/day, about 36 mg/kg/day, about 37 mg/kg/day, about 38 mg/kg/day, about 39 mg/kg/day, about 40 mg/kg/day, about 41 mg/kg/day, about 42 mg/kg/day, about 43 mg/kg/day, about 44 mg/kg/day, about 45 mg/kg/day, about 46 mg/kg/day, about 47 mg/kg/day, about 48 mg/kg/day, about 49 mg/kg/day, or about 50 mg/kg/day including all values and ranges therebetween. In some embodiments, the dose of CBD is split into two daily doses. In some embodiments, each daily dose is half of the total daily dose. In some embodiments, a patient administered a dose of 5 mg/kg/day is administered two daily doses of 2.5 mg/kg. In some embodiments, a patient administered a dose of 10 mg/kg/day is administered two daily doses of 5 mg/kg. In some embodiments, a patient administered a dose of 12.5 mg/kg/day is administered two daily doses of 6.25 mg/kg. In some embodiments, a patient administered a dose of 20 mg/kg/day is administered two daily doses of 10 mg/kg. In some embodiments, a patient administered a dose of 25 mg/kg/day is administered two daily doses of 12.5 mg/kg.

In some embodiments, CBD is administered to a patient at a starting dose of about 1 mg/kg/day, about 2 mg/kg/day, about 2.5 mg/kg/day, about 3 mg/kg/day, about 4 mg/kg/day, or about 5 mg/kg/day. In some embodiments, CBD is administered to a patient at a starting dose of about 5 mg/kg/day. In some embodiments, CBD is administered to a patient at a starting dose of about 5 mg/kg/day, wherein two daily doses of 2.5 mg/kg are administered. In some embodiments, CBD is administered to a patient at a starting dose of about 2.5 mg/kg/day, wherein two daily doses of 1.25 mg/kg are administered. In some embodiments, CBD is administered to a patient at a starting dose of about 1 mg/kg/day, wherein two daily doses of 0.5 mg/kg are administered.

In some embodiments, the daily dose of CBD is increased in increments ranging from about 1 mg/kg-5 mg/kg (e.g., about 1, 2, 2.5, 3, 4, or 5 mg/kg, including all values and ranges between these values). In some embodiments, the dose of CBD is increased from the starting dose in increments ranging about 0.5 mg/kg-5 mg/kg (e.g., about 0.5, 1, 1.25, 1.5, 2, 2.5, 3, 3.5, 3.75, 4, 4.5 or 5 mg/kg, including all values and ranged between these values). In some embodiments, the dose of CBD is increased by about 5 mg/kg increments, e.g., within one week. In some embodiments, the dose of CBD is increased from the starting dose by about 2.5 mg/kg increments. In some embodiments, the dose of CBD is increased from the starting dose of 5 mg/kg/day (e.g., 2.5 mg/kg twice daily) to 10 mg/kg/day (e.g., 5 mg/kg twice daily) after one week of treatment at 5 mg/kg/day. In some embodiments, the dose of CBD is increased from a dose of 10 mg/kg/day (e.g., 5 mg/kg twice daily) to 20 mg/kg/day (e.g., 10 mg/kg twice daily) by increasing the dose of CBD by 5 mg/kg/day each week until the 20 mg/kg/day dose is reached. In some embodiments, the dose of CBD is increased from 10 mg/kg/day to 20 mg/kg/day by increasing the dose of CBD by 5 mg/kg/day every other day, every 2 days, every 3 days, every 4 days, every 5 days, every 6 days, or every 7 days, until the 20 mg/kg/day dose is reached. In some embodiments, the dose of CBD is increased from a dose of 10 mg/kg/day (e.g., 5 mg/kg twice daily) to 25 mg/kg/day (e.g., 10 mg/kg twice daily) by increasing the dose of CBD by 5 mg/kg/day each week until the 25 mg/kg/day dose is reached. In some embodiments, the dose of CBD is increased from 10 mg/kg/day to 25 mg/kg/day by increasing the dose of CBD by 5 mg/kg/day every other day, every 2 days, every 3 days, every 4 days, every 5 days, every 6 days, or every 7 days, until the 25 mg/kg/day dose is reached. In some embodiments, the dose of CBD is increased to about 50 mg/kg/day by increasing the starting dose of CBD in increments ranging from 1 mg/kg/day to about 5 mg/kg/day every 2-7 days until the 50 mg/kg/day dose is reached.

In some embodiments, the starting dose of CBD is increased to a maintenance dose ranging from about 2 mg/kg/day to about 25 mg/kg/day, for example, about 2 mg/kg/day, about 3 mg/kg/day, about 4 mg/kg/day, about 5 mg/kg/day, about 6 mg/kg/day, about 7 mg/kg/day, about 8 mg/kg/day, about 9 mg/kg/day, about 10 mg/kg/day, about 11 mg/kg/day, about 12 mg/kg/day, about 13 mg/kg/day, about 14 mg/kg/day, about 15 mg/kg/day, about 16 mg/kg/day, about 17 mg/kg/day, about 18 mg/kg/day, about 19 mg/kg/day, about 20 mg/kg/day, about 21 mg/kg/day, about 22 mg/kg/day, about 23 mg/kg/day, about 24 mg/kg/day, or about 25 mg/kg/day. In some embodiments, the dose of CBD is increased to a maintenance dose of 10 mg/kg/day. In some embodiments, the dose of CBD is increased to a maintenance dose of 20 mg/kg/day. In some embodiments, the dose of CBD is increased to a maintenance dose of 25 mg/kg/day.

According to the FDA approved label for Epidiolex®, the dose of CBD can be increased “[a]fter one week . . . to a maintenance dosage of 5 mg/kg twice daily (10 mg/kg/day). Accordingly, the label teaches that a minimum dose of 10 mg/kg/day is needed for efficacy, and patients need to wait at least one week (i.e., a one week titration period) to safely and effectively titrate to the maintenance dose of 10 mg/kg/day. However, Applicant surprisingly and unexpectedly discovered that onset of effect of seizure reduction occurs within one week, e.g., at 2, 3, 4, 5, or 6 days—when patients are receiving 5 mg/kg/day. Accordingly, patients who need a higher maintenance dose to treat seizures (e.g., ranging from 10-25 mg/kg/day) may benefit from increasing the starting dose by about 1-5 mg/kg within one week of administering the starting dose, e.g., at 2, 3, 4, 5, or 6 days after administering the starting dose. These patients may also benefit from a more rapid dose escalation to reach maintenance doses of 20 or 25 mg/kg/day.

In some embodiments, the starting dose of CBD is increased to a maintenance dose of about 10 mg/kg/day within about 2, about 3, about 4, about 5, or about 6 days of administering the first starting dose. In some embodiments, the dose of about 10 mg/kg/day dose is increased by weekly increments of 5 mg/kg up to a maximum dose of about 20 mg/kg/day—i.e., the dose of about 10 mg/kg/day dose may be increased 5 mg/kg per week up to a maximum dose of about 20 mg/kg/day. In some embodiments, the maintenance dose of about 10 mg/kg/day dose is increased by about 5 mg/kg about every 2, 3, 4, 5, or 6 days to reach a maximum dose of, e.g., 20-25 mg/kg/day. In some embodiments, the maintenance dose of about 10 mg/kg/day dose is increased to a maximum dose of about 20 mg/kg/day within one week of the first administration of the maintenance dose of about 10 mg/kg/day, e.g., within about 3, about 4, about 5, or about 6 days. In some embodiments, the dose of about 20 mg/kg/day is increased to a maximum dose of about 25 mg/kg/day within one week (e.g., about 2, about 3, about 4, about 5, or about 6 days) of the first administration of the about 20 mg/kg/day dose.

In some embodiments, the disclosure provides methods of administering CBD (e.g., having a purity of at least 95% or 98% w/w) to treat patients with mild, moderate or severe hepatic impairment. Table 6 lists CBD doses for patients with mild, moderate or severe hepatic impairment.

TABLE 6
CBD Doses of Patients with Hepatic Impairment

| | | In Patients with LGS or DS | | In Patients |
|---|---|---|---|---|
| Hepatic | | | Maximum | with TSC |
| Impair- | Starting | Maintenance | Recommended | Maintenance |
| ment | Dosage | Dosage | Dosage | Dosage |
| Mild | 2.5 mg/kg | 5 mg/kg | 10 mg/kg | 12.5 mg/kg |
| | twice daily | twice daily | twice daily | twice daily |
| | (5 mg/kg/day) | (10 mg/kg/ | (20 mg/kg/ | (25 mg/kg/day) |
| | | day) | day) | |
| Mod- | 1.25 mg/kg | 2.5 mg/kg | 5 mg/kg | 6.25 mg/kg |
| erate | twice daily | twice daily | twice daily | twice daily |
| | (2.5 mg/ | (5 mg/kg/ | (10 mg/kg/ | (12.5 mg/ |
| | kg/day) | day) | day) | kg/day) |
| Severe | 0.5 mg/kg | 1 mg/kg | 2 mg/kg | 2.5 mg/kg |
| | twice daily | twice daily | twice daily | twice daily |
| | (1 mg/kg/day) | (2 mg/kg/ | (4 mg/kg/day) | (5 mg/kg/day) |
| | | day) | | |

In some embodiments, a patient with mild hepatic impairment is administered a starting dose of CBD of 5 mg/kg/day. In some embodiments, the dose of CBD administered is increased from the starting dose to a maintenance dose of about 10 mg/kg/day within about 2, about 3, about 4, about 5, or about 6 days. In some embodiments, the starting dose is increased by 5 mg/kg every 2, 3, 4, 5, 6 or 7 days.

In some embodiments, a patient with moderate hepatic impairment is administered a starting dose of CBD of 2.5 mg/kg/day. In some embodiments, the dose of CBD administered is increased from the starting dose to a maintenance dose of about 5 mg/kg/day within about 2, about 3, about 4, about 5, or about 6 days. In some embodiments, the starting dose is increased by 2.5 mg/kg every 2, 3, 4, 5, 6, or 7 days.

In some embodiments, a patient with severe hepatic impairment is administered a starting dose of CBD of 1 mg/kg/day. In some embodiments, the dose of CBD administered is increased from the starting dose to a maintenance dose of about 2 mg/kg/day within about 2, about 3, about 4, about 5, or about 6 days. In some embodiments, the starting dose is increased by 1 mg/kg every 2, 3, 4, 5, 6, or 7 days.

### Preparation of Highly Purified Cbd Extract

The following describes the production of the highly-purified (>98% w/w) cannabidiol extract which has a known and constant composition which was used for the expanded access trials described in Examples below.

In summary the drug substance used in the trials is a liquid carbon dioxide extract of high-CBD containing chemotypes of Cannabis sativa L. which had been further purified by a solvent crystallization method to yield CBD. The crystallisation process specifically removes other cannabinoids and plant components to yield greater than 95% CEO w/w, typically greater than 98% w/w.

The Cannabis sativa L. plants are grown, harvested, and processed to produce a botanical extract (intermediate) and then purified by crystallization to yield the CBDe (drug substance).

The plant starting material is referred to as Botanical Raw Material (ERM); the botanical extract is the intermediate; and the active pharmaceutical ingredient (API) is Ce, the drug substance.

Both the botanical starting material and the botanical extract are controlled by specifications. The drug substance specification is described in Table 7 below.

TABLE 7
CBD Specification

| Test | Test Method | Limits |
|---|---|---|
| Appearance | Visual | Off-white/pale yellow crystals |
| Identification A | HPLC-UV | Retention time of major peak |
| | | corresponds to certified CBD Reference |
| | | Standard |
| Identification B | GC-FID/MS | Retention time and mass spectrum of |
| | | major peak corresponds to certified |
| | | CBD Reference Standard |
| Identification C | FT-IR | Conforms to reference spectrum for |
| | | certified CBD Reference Standard |
| Identification D | Melting Point | 65-67° C. |
| Identification E | Specific Optical Rotation | Conforms with certified CBD Reference |
| | | Standard; −110° to −140° (in 95% |
| | | ethanol) |
| Total Purity | Calculation | ≥98.0% |
| Chromatographic Purity 1 | HPLC-UV | ≥98.0% |
| Chromatographic Purity 2 | GC-FID/MS | ≥98.0% |
| Other Cannabinoids: | HPLC-UV | NMT 0.15% w/w |
| CBDA | | NMT 1.0% w/w |
| CBDV | | NMT 0.15% w/w |
| Δ⁹ THC | | NMT 0.5% w/w |
| CBD-C4 | | |
| Residual Solvents: | GC | NMT 0.5% w/w |
| Alkane | | NMT 0.5% w/w |
| Ethanol | | |
| Residual Water | Karl Fischer | NMT 1.0% w/w |

NMT—Not more than

The purity of the CEO drug substance achieved is greater than 98%. The other cannabinoids which may occur in the extract are: CBDA, CBDV, CBD-C4 and THC.

Distinct chemotypes of Cannabis sativa L. plant have been produced to maximize the output of the specific chemical constituents, the cannabinoids. One type of plant produces predominantly CBD. Only the (−)-trans isomer occurs naturally, furthermore during purification the stereochemistry of CBD is not affected.

### Production of the Intermediate

An overview of the steps to produce a botanical extract, the intermediate, are as follows:
1. Growing
2. Decarboxylation
3. Extraction No. 1—using liquid CO₂
4. Extraction No. 2—‘winterization’ using ethanol
5. Filtration
6. Evaporation

High CBD chemovars were grown, harvested and dried and stored in a dry room until required. The botanical raw material (BRM) was finely chopped using an Apex mill fitted with a 1 mm screen. The milled BRM was stored in a freezer for up to 3 months prior to extraction.

Decarboxylation of CBDA to CBD was carried out using a large Heraeus tray oven. The decarboxylation batch size in the Heraeus is approximately 15 Kg. Trays were placed in the oven and heated to 105° C.; the BRM took 96.25 minutes to reach 105° C. Held at 105° C. for 15 Minutes. Oven then set to 150° C.; the BRM took 75.7 minutes to reach 150° C.; BRM held at 150° C. for 130 Minutes. Total time in the oven was 380 Minutes, including 45 minutes cooling and 15 Minutes venting.

Extraction No 1 was performed using liquid CO₂ at 60 bar/10° C. to produce botanical drug substance (BDS) which was used for crystallisation to produce the test material.

The crude CBD BDS was winterized in Extraction No 2 under standard conditions (2 volumes of ethanol at minus 20° C. for around 50 hours). The precipitated waxes were removed by filtration and the solvent evaporated using the rotary evaporator (water bath up to 60° C.) to yield the BDS.

### Production of the Drug Substance

The manufacturing steps to produce the drug substance from the intermediate botanical extract are as follows:
1. Crystallization using C5-C12 straight chain or branched alkane (e.g., pentane or hexane)
2. Filtration
3. Optional recrystallization from C5-C12 straight chain or branched alkane (e.g., pentane or hexane)
4. Vacuum drying

Intermediate botanical extract (12 kg) produced using the methodology above was dispersed in C5-C12 straight chain or branched alkane (9000 ml, 0.75 vols) in a 30 litre stainless steel vessel.

The mixture was manually agitated to break up any lumps and the sealed container then placed in a freezer for approximately 48 hours.

The crystals were isolated by vacuum filtration, washed with aliquots of cold C5-C12 straight chain or branched alkane (total 12000 ml), and dried under a vacuum of <10 mb at a temperature of 60° C. until dry before submitting the drug substance for analysis. The dried product was stored in a freezer at minus 20° C. in a pharmaceutical grade stainless steel container, with FDA food grade approved silicone seal and clamps.

### Production of the Drug Product

The drug product is presented as an oral solution. The oral solution presentation contains 25 mg/ml or 100 mg/ml CBD, with the excipients sesame oil, ethanol, sucralose and flavouring. Two product strengths are available to allow dose titration across a wide dose range.

The 25 mg/ml solution is appropriate at lower doses and the 100 mg/ml solution at higher doses.

The drug product formulation is as described in Table 8 below:

TABLE 8
Drug Product specification

| | Qualitative | | Reference to |
|---|---|---|---|
| Component | Composition | Function | Quality Standard |
| Cannabidiol (CBD) | 25 mg/ml or | Active | In-house |
| | 100 mg/ml | | |
| Anhydrous ethanol | 79.0 mg/ml* | Excipient | Ph. Eur. |
| Sucralose | 0.5 mg/ml | Sweetener | In-house |
| Strawberry flavouring | 0.2 mg/ml | Flavouring | In-house |
| Sesame oil | q.s to 1.0 ml | Excipient | Ph. Eur. |

The drug substance, CBD is insoluble in water. Sesame oil was selected as an excipient to solubilize the drug substance.

A sweetener and fruit flavouring are required to improve palatability of the sesame oil solution.

Ethanol was required to solubilize the sweetener and the flavouring.

The composition can be substantially equivalent, by which is meant the functional ingredients can vary from the qualitative composition specified in Table 8 by an amount of up to 10%.

Example 1 below describes the use of a highly purified cannabis extract comprising cannabidiol (CBD) in an expanded access treatment program in children with TRE.

### Example 1: Efficacy of Cannabidiol at Reducing Seizures in 1-2 Year Olds with Dravet Syndrome (DS), Lennox-Gastaut Syndrome (LGS), and Tuberous Sclerosis Complex (TSC)

### Materials and Methods

Patients aged 1-2 years old with Dravet Syndrome (DS), Lennox-Gastaut Syndrome (LGS), or Tuberous Sclerosis Complex (TSC) were administered a highly purified extract of cannabidiol (CBD) obtained from a cannabis plant or placebo.

The highly purified CBD extract (greater than 98% CBD w/w) was administered in a formulation described herein at a starting dose of 5 mg/kg/day in addition to their baseline anti-epileptic drug (AED) regimen.

The starting dose of the patients was increased from 5 mg/kg/day to 10 mg/kg/day after one week of administering the first dose of CBD. The dose was subsequently increased to 20-50 mg/kg/day after about two-four weeks of administering the first dose of CBD.

All patients were taking at least two concomitant anti-epileptic drugs. These included clobazam; levetiracetam; topiramate; vigabatrin; phenobarbital; and lacosamide.

Prior to treatment with CBD, the number of seizures for each patient at baseline was recorded. During the course of treatment, the number of seizures that each patient experienced per day was recorded. Table 9 provides information on the demographics, seizure type, and concomitant anti-epileptic drugs (AED) for each patient that was administered the highly purified extract of CBD.

TABLE 9
Patient demographics, seizure type and concomitant medication

| | | Age | | | Concomitant |
|---|---|---|---|---|---|
| Patient ID # | Aetiology | (years) | Sex | Seizure type | AEDs |
| 1 (LCH09) | Dravet Syndrome | 1.70 | Male | Tonic clonic; | Clobazam, |
| | | | | Clonic; | topiramate, |
| | | | | Focal with impairment | phenobarbital |
| 2 (PAT27) | Lennox-Gastaut | 1.00 | Female | Clonic; | Levetiracetam, |
| | Syndrome | | | Atonic; | vigabatrin |
| | | | | Absence; | |
| | | | | Tonic | |
| 3 (B39) | Tuberous | 1.82 | Male | Focal with impairment; | Clobazam, |
| | Sclerosis | | | Focal without | levetiracetam, |
| | Syndrome | | | impairment; | vigabatrin, |
| | | | | Focal with secondary | lacosamide |
| | | | | generalisation | |

### Results

Tables 10A-C show the dose of CEO and seizure frequency for each patient over the course of the study. Patient 1, who was diagnosed with Dravet Syndrome, experienced a 100% reduction in clonic seizures and focal seizures with impairment over the treatment period. Patient 2, who was diagnosed with Lennox-Gastaut Syndrome, experienced a 100% reduction in clonic, atonic and absence seizures over the treatment period. Patient 3, who was diagnosed with Tuberous Sclerosis Complex, experienced a 100% reduction in focal seizures with impairment and focal seizures with secondary generalisation seizures over the treatment period.

This study shows the efficacy of CEO for reducing the frequency of many seizure types, including, clonic, atonic, absence seizures, focal seizures with impairment and focal seizures with secondary generalisation. CEO is especially efficacious at reducing clonic seizures and focal seizures with impairment. Both patients who suffered from clonic seizures (patients 1 and 2) and both patients who suffered from focal seizures with impairment (patients 1 and 3) experienced complete relief from the aforementioned seizures after treatment with CEO.

TABLE 10A
Seizure frequency data for Patient 1
Patient 1

| | Seizure Type | | | |
|---|---|---|---|---|
| | | | Focal with | Dose CBD |
| Time | Clonic | Tonic-clonic | impairment | (mg/kg/day) |
| Baseline | 4.0 | 3.0 | 3.0 | — |
| 2 weeks | 6.0 | 4.0 | 0.0 | 11.7 |
| 4 weeks | 4.0 | 2.0 | 0.0 | 19.2 |
| 8 weeks | 2.0 | 0.0 | 0.0 | 24.3 |
| 16 weeks | 2.6 | 0.0 | 0.0 | 25.4 |
| 24 weeks | 0.8 | 1.6 | 0.0 | 24.8 |
| 36 weeks | 0.0 | 6.9 | 0.0 | 23.0 |
| 48 weeks | 0.0 | 6.5 | 0.0 | 22.6 |

TABLE 10B
Seizure frequency data for Patient 2
Patient 2

| | Seizure Type | | | | Dose CBD |
|---|---|---|---|---|---|
| Time | Clonic | Tonic | Atonic | Absence | (mg/kg/day) |
| Baseline | 200.0 | 4.0 | 300.0 | 120.0 | 5.0 |
| 4 weeks | 64.0 | 116.0 | 388.0 | 116.0 | 25.0 |
| 8 weeks | 40.0 | 48.0 | 564.0 | 78.0 | 50.0 |
| 12 weeks | 40.0 | 116.0 | 300.0 | 140.0 | 49.6 |
| 24 weeks | 40.0 | 116.0 | 280.0 | 140.0 | 49.0 |
| 36 weeks | 40.0 | 116.0 | 280.0 | 140.0 | 48.6 |
| 60 weeks | 0.0 | 1.0 | 1.0 | 40.0 | 3.7 |
| 72 weeks | 0.0 | 20.0 | 0.0 | 0.0 | 50.0 |

TABLE 10C
Seizure frequency data for Patient 3
Patient 3

| | Seizure Type | | | |
|---|---|---|---|---|
| | | | Focal with | |
| | Focal with | Focal without | secondary | Dose CBD |
| Time | impairment | impairment | generalisation | (mg/kg/day) |
| Baseline | 25.4 | 0.0 | 63.0 | — |
| 2 weeks | 20.0 | 0.0 | 50.0 | 10.0 |
| 4 weeks | 22.0 | 0.0 | 61.0 | 20.0 |
| 8 weeks | 15.4 | 0.0 | 109.1 | 15.0 |
| 16 weeks | 0.6 | 0.0 | 46.2 | 15.0 |
| 24 weeks | 0.0 | 0.0 | 58.1 | 13.0 |
| 36 weeks | 0.0 | 0.0 | 0.0 | 13.0 |
| 48 weeks | 0.0 | 0.0 | 14.8 | 13.0 |
| 60 weeks | 71.3 | 0.0 | 0.0 | 11.5 |
| 84 weeks | 62.5 | 0.0 | 0.0 | 27.0 |
| 96 weeks | 14.8 | 0.0 | 0.0 | 22.0 |
| 108 weeks | 0.3 | 10.8 | 0.0 | 13.0 |
| 120 weeks | 0.0 | 11.4 | 0.0 | 29.0 |
| 144 weeks | 0.0 | 9.8 | 0.0 | 35.0 |

1. A method of treating seizures in a patient in need thereof, comprising administering to the patient a cannabidiol (CBD) drug substance, wherein the CBD drug substance has a purity of at least 95% (w/w) CBD, wherein the patient is about 1-2 years of age, and wherein the CBD is administered at a dose ranging from about 5 mg/kg/day to about 50 mg/kg/day.

2. The method of claim 1, wherein the CBD is administered at a dose of 25 mg/kg/day.

3. The method of claim 1, wherein the CBD is administered at a dose of 20 mg/kg/day.

4. The method of claim 1, wherein the CBD is administered at a dose of 15 mg/kg/day.

5. The method of claim 1, wherein the CBD is administered at a dose of 10 mg/kg/day.

6. The method of any of claims 1 to 5, wherein the administering treats generalized seizures or focal seizures.

7. The method of claim 6, wherein the generalized seizures comprise tonic, atonic, tonic-clonic, or absence seizures.

8. The method of claim 6, wherein the focal seizures are focal motor seizures without impairment of consciousness or awareness, focal seizures with impairment of consciousness or awareness, or focal seizures which evolve to secondary generalized seizures.

9. The method of any of claims 1 to 8, wherein the administering reduces the total number of seizures.

10. The method of claim 9, wherein the number of seizures is reduced by at least 50% compared to the number of seizures experienced during a baseline period before CBD was administered.

11. The method of claim 10, wherein the seizures are generalized seizures or focal seizures.

12. The method of claim 11, wherein the generalized seizures comprise tonic, atonic, tonic-clonic, or absence seizures.

13. The method of claim 11, wherein the focal seizures are focal motor seizures without impairment of consciousness or awareness, focal seizures with impairment of consciousness or awareness, or focal seizures which evolve to secondary generalized seizures.

14. The method of any of claims 9 to 13, wherein the CBD is administered at a dose of 10 mg/kg/day.

15. The method of any of claims 9 to 13, wherein CBD is administered at a dose of about 20 mg/kg/day.

16. The method of any of claims 9 to 13, wherein CBD is administered at a dose of about 25 mg/kg/day.

17. The method of any of claims 1 to 16, wherein the CBD is present as a synthetic compound.

18. The method of any of claims 1 to 16, wherein the CBD is present in a highly purified CBD extract.

19. The method of any of claims 1 to 18, wherein the CBD drug substance has a purity of at least 98% (w/w) CBD.

20. The method of any of claims 1 to 19, wherein the patient is suffering from Lennox-Gastaut Syndrome (LGS); Tuberous Sclerosis Complex (TSC); Dravet Syndrome (DS); Doose Syndrome; Aicardi syndrome; CDKL5 or Dup15q.

21. The method of claim 1, wherein the patient suffers from Tuberous Sclerosis Complex.

22. The method of claim 1, wherein the patient suffers from Dravet Syndrome.

23. The method of claim 1, wherein the patient suffers from Lennox-Gastaut Syndrome.

24. The method of claim 21, wherein CBD is administered at a dose of about 25 mg/kg/day.

25. The method of claim 22, wherein the CBD is administered at a dose of 10 mg/kg/day.

26. The method of claim 22, wherein CBD is administered at a dose of about 20 mg/kg/day.

27. The method of claim 23, wherein the CBD is administered at a dose of 10 mg/kg/day.

28. The method of claim 23, wherein CBD is administered at a dose of about 20 mg/kg/day.
